(19) United States
(12) Patent Application Publication
(10) Pub. No.: US 2020/0078172 A1
(43) Pub. Date: Mar. 12, 2020

(54) DEVICES AND METHODS FOR TRANSCATHETER RETRIEVAL OF MECHANICAL HEART VALVE LEAFLETS

(71) Applicant: CEDARS-SINAI MEDICAL CENTER, Los Angeles, CA (US)

(72) Inventors: Ali Khoynezhad, Manhattan Beach, CA (US); Paul Philipp Heinisch, Munich (DE); Ralf Brecht, Munich (DE); Selena Akra, Olching (DE)

(73) Assignee: CEDARS-SINAI MEDICAL CENTER, Los Angeles, CA (US)

(21) Appl. No.: 16/389,661

(22) Filed: Apr. 19, 2019

Related U.S. Application Data
(63) Continuation of application No. 14/915,133, filed on Feb. 26, 2016, now abandoned.
(60) Provisional application No. 61/872,171, filed on Aug. 30, 2013.

(51) Int. Cl. A61F 2/24; A61B 17/221

(52) U.S. Cl. CPC A61F 2/2433; A61B 17/221; A61B 2017/22097; A61B 2017/2215; A61F 2/2409; A61F 2250/0003

(57) ABSTRACT

The present invention teaches apparatuses and methods for transcatheter removal of mechanical heart valve leaflets from a subject. The invention also teaches methods for subsequent transcatheter placement of a bioprosthesis within the annulus that remains after the leaflets are removed. Advantageously, the devices and methods of the present invention can be used without a heart-lung machine.

### CROSS REFERENCE TO RELATED APPLICATIONS

This application is a continuation of U.S. application Ser. No. 14/915,133, filed Feb. 26, 2016, which is a National Phase of International Application No. PCT/US2014/053727, filed Sep. 2, 2014, which designated the U.S. and that International Application was published under PCT Article 21(2) in English, which claims priority under 35 U.S.C. § 119(e) to U.S. Provisional Application No. 61/872,171, filed Aug. 30, 2013, the contents of which are incorporated herein by reference in their entirety.

### FIELD OF THE INVENTION

The present invention generally relates to apparatuses and methods for surgical heart valve procedures.

### BACKGROUND

The following description includes information that may be useful in understanding the present invention. It is not an admission that any of the information provided herein is prior art or relevant to the presently claimed invention, or that any publication specifically or implicitly referenced is prior art.

There are currently over two million patients with mechanical heart valves. If a patient needs to have a biological valve instead of a mechanical valve, a high-risk open-heart operation has to be performed to remove the mechanical valve and implant a biological one. There is a need in the art for safer and more efficient catheter-based devices and methods for removing a mechanical heart valve (or portions thereof) and replacing it with a biological or bioprosthetic valve.

### SUMMARY OF THE INVENTION

In various embodiments, the invention teaches an apparatus for transcatheter removal of mechanical heart valve leaflets from a subject, including: a catheter, including: a first catheter end; a second catheter end; and an elongated catheter body; a sheath, including: a first sheath end; a second sheath end; and an elongated sheath body; a leaflet capturing component, including: an attaching end for attaching to a region near the first catheter end; an elongated leaflet capturing body; and a sliding end; wherein (1) the catheter, sheath and leaflet capturing component are situated concentrically, such that the sheath is the outermost component and the catheter is the innermost component; (2) the sliding end is configured to traverse the elongated catheter body; and (3) when the sliding end is advanced in the direction of the attaching end, a section of the leaflet capturing component is extended beyond the first sheath end, thereby forming a basket. In certain embodiments, the leaflet capturing component includes mesh. In some embodiments, the mesh is made of a material selected from the group consisting of nitinol, semicompliant platinum, nickel-chromium, and combinations thereof. In certain embodiments, the apparatus includes an inflatable balloon. In certain embodiments, the inflatable balloon is located between the attaching end of the leaflet capturing component and the first catheter end. In certain embodiments, the inflatable balloon is asymmetrical when inflated. In certain embodiments, the inflatable balloon has an asymmetrical half dumbbell shape when inflated. In certain embodiments, the apparatus further includes a means for inflating the balloon. In certain embodiments, the apparatus includes a means for causing the leaflet capturing component to form a basket. In certain embodiments, the catheter is telescoping, such that its overall length can be adjusted.

In various embodiments, the invention teaches a system, including: (a) an apparatus for transcatheter removal of mechanical heart valve leaflets from a subject, that includes: a catheter, including: a first catheter end; a second catheter end; and an elongated catheter body with an elongated lumen traversing the long axis of said elongated catheter body; a sheath, including a first sheath end; a second sheath end; and an elongated sheath body; a leaflet capturing component, including: an attaching end for attaching to a region near the first catheter end; an elongated leaflet capturing body; and a sliding end; wherein (1) the catheter, sheath and leaflet capturing component are situated concentrically, such that the sheath is the outermost component and the catheter is the innermost component; (2) the sliding end is configured to traverse the elongated catheter body; and (3) when the sliding end is advanced in the direction of the attaching end, a section of the leaflet capturing component is extended beyond the first sheath end, thereby forming a basket; and (b) a guidewire configured to traverse the elongated lumen of the catheter body.

In various embodiments, the invention teaches a method that includes introducing an apparatus described herein above into the heart of a subject having a mechanical heart valve, wherein the mechanical heart valve includes mechanical heart valve leaflets attached to a mechanical heart valve annulus; introducing a balloon of a balloon catheter between the mechanical heart valve leaflets of the mechanical heart valve; inflating the balloon until the mechanical heart valve leaflets are separated from the annulus of the mechanical heart valve; forming a basket with the leaflet capturing component of the apparatus by advancing the sliding end of the leaflet capturing component in the direction of the attaching end of the leaflet capturing component; enveloping the separated mechanical heart valve leaflets with the basket; withdrawing the basket in the direction of the second end of the catheter, thereby substantially containing the mechanical heart valve leaflets within the basket; and removing the apparatus and the mechanical heart valve leaflets from the subject. In certain embodiments, the apparatus is introduced into the subject's heart through a blood vessel. In certain embodiments, the apparatus is introduced into the subject's heart transapically. In certain embodiments, the method further includes placing a replacement heart valve in the mechanical heart valve annulus by using a balloon catheter, after the mechanical heart valve leaflets have been removed. In certain embodiments, the replacement heart valve is a bioprosthetic valve. In certain embodiments, the apparatus and the balloon catheter are inserted into the subject's body such that they approach the mechanical heart valve from opposite directions. In some embodiments, the apparatus and the balloon catheter are inserted into the subject's body such that they each approach the mechanical heart valve from the same direction. In certain embodiments, the mechanical heart valve is located in the position of a heart valve selected from the group consisting of an aortic valve, a mitral valve, a pulmonic valve and a tricuspid valve.

In various embodiments, the invention teaches a method that includes introducing an apparatus that includes a balloon, as described above, into the heart of a subject having a mechanical heart valve that includes mechanical heart valve leaflets attached to a mechanical heart valve annulus; introducing the balloon of the apparatus between the mechanical heart valve leaflets of the mechanical heart valve; inflating the balloon of the apparatus until the mechanical heart valve leaflets are separated from the annulus of the mechanical heart valve; forming a basket with the leaflet capturing component of the apparatus by advancing the sliding end of the leaflet capturing component in the direction of the attaching end of the leaflet capturing component; enveloping the mechanical heart valve leaflets with the basket; withdrawing the basket in the direction of the second end of the catheter, thereby substantially containing the mechanical heart valve leaflets within the basket; deflating the balloon; and removing the apparatus and the mechanical heart valve leaflets from the subject. In certain embodiments, the mechanical heart valve is located in the position of a heart valve selected from the group consisting of an aortic valve, a mitral valve, a pulmonic valve and a tricuspid valve. In some embodiments, the method further includes placing a replacement heart valve in the mechanical heart valve annulus by utilizing a balloon catheter, after the mechanical heart valve leaflets have been removed. In certain embodiments, the replacement heart valve is a bioprosthetic valve.

In various embodiments, the invention teaches a kit that includes (a) an apparatus for transcatheter removal of mechanical heart valve leaflets from a subject, including: a catheter, including: a first catheter end; a second catheter end; and an elongated catheter body with an elongated lumen traversing the long axis of said elongated catheter body; a sheath, including: a first sheath end; a second sheath end; and an elongated sheath body; a leaflet capturing component, including: an attaching end for attaching to a region near the first catheter end; an elongated leaflet capturing body; and a sliding end; wherein (1) the catheter, sheath and leaflet capturing component are situated concentrically, such that the sheath is the outermost component and the catheter is the innermost component; (2) the sliding end is configured to traverse the elongated catheter body; and (3) when the sliding end is advanced in the direction of the attaching end, a section of the leaflet capturing component is extended beyond the first sheath end, thereby forming a basket; (b) a guidewire configured to traverse the elongated lumen of the catheter body; and optionally (c) instructions for the use of the apparatus for removing mechanical heart valve leaflets from a subject.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 depicts, in accordance with an embodiment of the invention, a device in the process of capturing mechanical heart valve leaflets that have been separated from the annulus of a mechanical valve.

FIG. 2 depicts, in accordance with an embodiment of the invention, a device with an integrated balloon. The device is shown separating the mechanical heart valve leaflets from the valve annulus and then capturing the leaflets in a retrieval basket.

FIG. 3 depicts, in accordance with an embodiment of the invention, a device with a telescoping balloon component. The balloon component is shown extending from the end of the device and into the mechanical valve where it separates the mechanical valve leaflets from the valve annulus upon inflation.

FIG. 4 depicts, in accordance with an embodiment of the invention, a balloon catheter approaching the valve from one direction, and a leaflet capturing device approaching the valve from the opposite direction. The leaflet capturing device includes strings for manipulating the shape of the mesh basket.

FIG. 5 depicts, in accordance with an embodiment of the invention, two possible approaches (transapical and through the aorta) to reach the mechanical heart valve that is to be replaced using the inventive methods and devices.

FIG. 6 depicts, in accordance with an embodiment of the invention, a balloon catheter making a transapical approach to a mechanical aortic heart valve. A leaflet capturing device with a mesh basket is shown approaching the valve from the ascending aorta and capturing the leaflets once they have been separated.

FIG. 7 depicts, in accordance with an embodiment of the invention, a balloon catheter approaching a mechanical aortic heart valve from the ascending aorta, and a leaflet capturing device making a transapical approach.

FIG. 8 depicts, in accordance with an embodiment of the invention, placing a replacement heart valve in a mechanical valve annulus using a balloon catheter.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Szycher's Dictionary of Medical Devices (1995); and Iaizzo, Handbook of Cardiac Anatomy, Physiology, and Devices (2009), may provide useful guidance to many of the terms and phrases used herein.

In some embodiments, properties such as dimensions, shapes, relative positions, and so forth, used to describe and claim certain embodiments of the invention are to be understood as being modified by the term “about.”

One skilled in the art will recognize many methods and materials similar or equivalent to those described herein, which could be used in the practice of the present invention. Indeed, the present invention is in no way limited to the methods and materials described. For purposes of the present invention, certain terms are defined below.

“Mammal” as used herein refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. The term does not denote a particular age or sex. Thus, adult and newborn subjects, whether male or female, are intended to be included within the scope of this term. In certain embodiments described herein, the inventive devices and methods are configured for use in humans. One of skill in the art would readily appreciate that the devices and methods described herein could be customized for use in almost any animal in which a mechanical heart valve is implanted.

By way of additional background, the “valve-in-valve” concept of implanting a transcatheter valve into a biological prosthetic heart valve was originally introduced in 2008 by Dr. Thomas Walther and coworkers. While that “valve-in-valve” procedure has proven very useful, it can only be implemented in patients that have a biological prosthesis. Unfortunately, a relatively large number of patients are in need of a procedure to replace a mechanical heart valve with a bioprosthetic valve. These patients generally fall into two categories, (1) those with a malfunctioning mechanical heart valve (due to pannus and/or significant hemolysis) and (2) those with contraindication for systemic anticoagulation (e.g. patients with a cerebral bleed, active gastrointestinal bleeding, elderly patients at risk for falling, young women desiring to have a pregnancy and the like). Importantly, the perioperative mortality for redo/replacement procedures involving the aortic or mitral valve is reported to be from three to twenty-five percent.

Recognizing this problem, the inventors developed devices and methods that allow for much safer and more efficient approaches to replacing a mechanical heart valve without the need for a high-risk redo open-heart operation that requires a heart-lung machine. Although there are a number of different devices described herein, each facilitates removal of mechanical heart valve leaflets from a subject, once they have been separated from a mechanical heart valve annulus. After the leaflets have been removed, a new valve can be placed inside the remaining annulus of the mechanical heart valve via a transcatheter procedure.

### Central Components

Certain features are common to each of the devices described herein. First, each device includes a catheter, which includes a leading end, an elongated body, and a trailing end. The leading end of the catheter is the first part of the device to be inserted into a patient when the procedures described herein are performed. The next common component is a mechanism for capturing the leaflets of a mechanical heart valve once they have been separated from a mechanical heart valve annulus. Another common feature is a sheath for housing both the catheter and the mechanism for capturing the mechanical heart valve leaflets. The sheath helps to achieve a compact form, and facilitates entry of the device into a patient and withdrawal of the device from a patient, when the procedures described herein are performed. Finally, a central lumen can be incorporated into each of the devices described herein, so that the devices can be fitted over a guidewire.

In various embodiments, the catheter is made of a material that may include, but is in no way limited to plastic, hydrophilic material, polyester, and the like. In certain embodiments, the catheter can be from 20-140 cm long, or 40-120 cm long, or 60-100 cm long, or 80-90 cm long. with a diameter ranging from 10-26 F. In some embodiments, the sheath of the device is cylindrical. One of skill in the art would readily appreciate that alternative shapes could also be used. In some embodiments, the sheath of the device is made of a material that may include, but is in no way limited to, plastic, hydrophilic material, polyester, and the like. In some embodiments, the length of the sheath is sufficient to cover all or a substantial portion of the leaflet capturing component.

### Leaflet Capturing Components

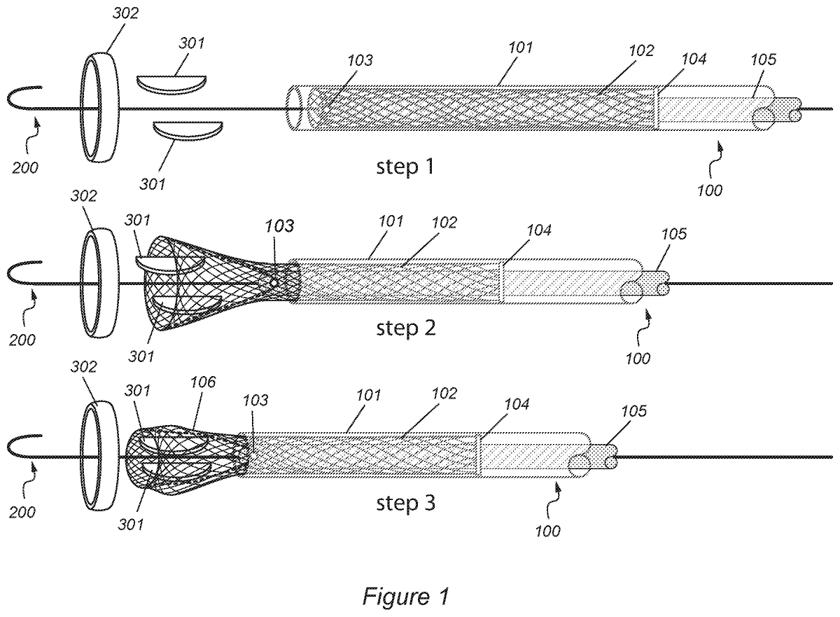

In some embodiments described herein, the leaflet capturing component is capable of fitting between the sheath and catheter body when the device is introduced into a patient. In some embodiments, the leaflet capturing component is formed by a substantially cylindrically shaped material that includes an attaching end, an elongated body, and a sliding end. As demonstrated in FIG. 1, the attaching end 103 of the capturing component can be attached to the catheter near its leading end, and the body 102 and sliding end 104 of the capturing component extends towards the trailing end of the catheter. As demonstrated in FIG. 1 (steps 1-3), in some embodiments the elongated body 102 and sliding end 104 of the capturing component are configured to slide along the elongated body of the catheter 105. The attaching end of the leaflet capturing component can be attached to the catheter by any of a number of ways. Merely by way of non-limiting examples, the attaching end can be glued, embedded, looped/stitched through the catheter body, or fastened to the catheter body by any other fastening means known in the art.

In some embodiments, the capturing component is 5-100% of the overall device length. In some embodiments, the capturing component is 10-80% of the overall device length. In some embodiments, the capturing component is 20-70% of the overall device length. In some embodiments, the capturing component is 30-60% of the overall device length. In some embodiments, the capturing component is between 40-50% of the overall device length. In certain embodiments, the outer sheath is of a greater length than the capturing component.

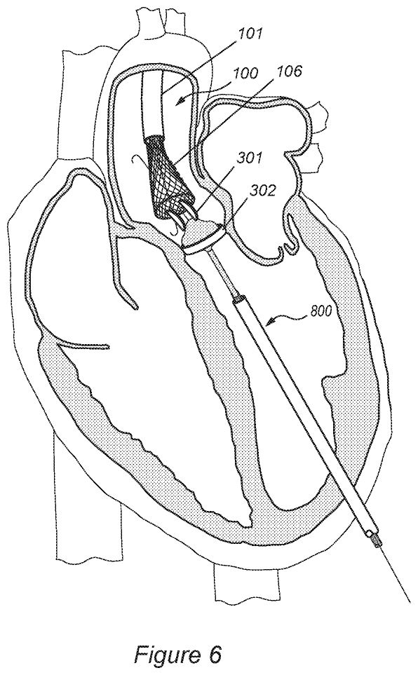

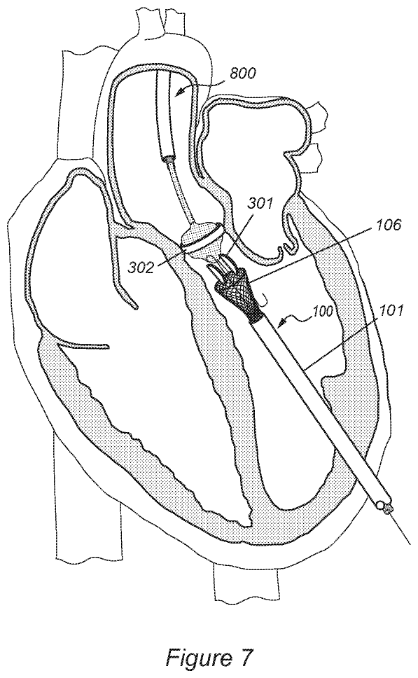

In various embodiments, the capturing component is a mesh. In some embodiments, the mesh is of a type that can be manipulated to adopt various shapes. In some embodiments, the mesh can be manipulated to adopt a basket shape. In certain embodiments, the mesh is capable of maintaining memory (“memory mesh” hereafter). In some embodiments, the memory mesh is capable of conforming to an approximately cylindrical shape when compressed between the body of the catheter and the sheath, yet it adopts another predetermined shape when a section of sufficient length is advanced beyond an end of the sheath. Merely by way of example, as shown in FIG. 1, a section of the memory mesh 106 can be configured to adopt a cone-like shape, thereby essentially forming a basket, when it is released from the sheath. In various embodiments, the memory mesh is configured to form a basket of a small enough diameter such that it can fit within the desired region of a subject's heart described herein. One of skill in the art would readily appreciate that the dimensions of the inventive apparatus, and the components thereof, could be readily configured to suit the particular environment in which the apparatus/device is intended to be used, and may therefore be different for differently sized subjects and anatomical locations within the heart. In some embodiments, the diameter of the basket is such that it allows the apparatus to be positioned proximal to or in a mechanical heart valve of a patient. In some embodiments, the basket of the apparatus is configured to be positioned proximal to (on either side of) or in one or more of an aortic valve, a mitral valve, a pulmonic valve, or a tricuspid valve. In certain embodiments, the basket (mesh or otherwise) formed by the inventive apparatus can be any size that is appropriate to the anatomical location in which it is intended to be used, so long as it is large enough to accommodate the mechanical heart valve leaflet(s) it is intended to retrieve. In an embodiment, the basket is of a sufficient size to accommodate two or more mechanical heart valve leaflets of an aortic valve, as demonstrated in FIGS. 6 and 7. Importantly, although only an aortic valve and the approach thereto is specifically shown in the figures, one of skill in the art would readily appreciate that the device can be used to remove mechanical heart valve leaflets of the mitral valve, pulmonic valve, or tricuspid valve, as well as any other mechanical valve with leaflets positioned anywhere in the body. In some embodiments, the diameter of the widest section of the basket formed by the memory mesh is slightly smaller than the diameter of a mechanical heart valve annulus when the basket is formed. In some embodiments, the diameter of the widest section of the basket is approximately equal to the diameter of a mechanical heart valve annulus when the basket is formed. In some embodiments, the basket is of a larger diameter than a mechanical heart valve annulus when the basket is formed. In some embodiments, the diameter of the basket is approximately equal to the diameter of the lumen extending in either direction proximal to the mechanical heart valve of the patient. Merely by way of example, the diameter of the basket can be approximately equal to the diameter of the portion of the left ventricle proximal to the mechanical heart valve. Alternatively, the diameter of the basket can be approximately equal to that of the portion of the ascending aorta proximal to the mechanical heart valve. In some embodiments, the basket is sized to fit any valve described herein, or lumen associated therewith, according to the relative proportions described for interacting with the aortic valve above.

The apparatus/device as a whole can be configured with appropriate dimensions to be inserted into the left ventricle, right ventricle, left atrium, right atrium, ascending aorta, superior vena cava, or inferior vena cava, such that it can approach any valve described herein from either side thereof.

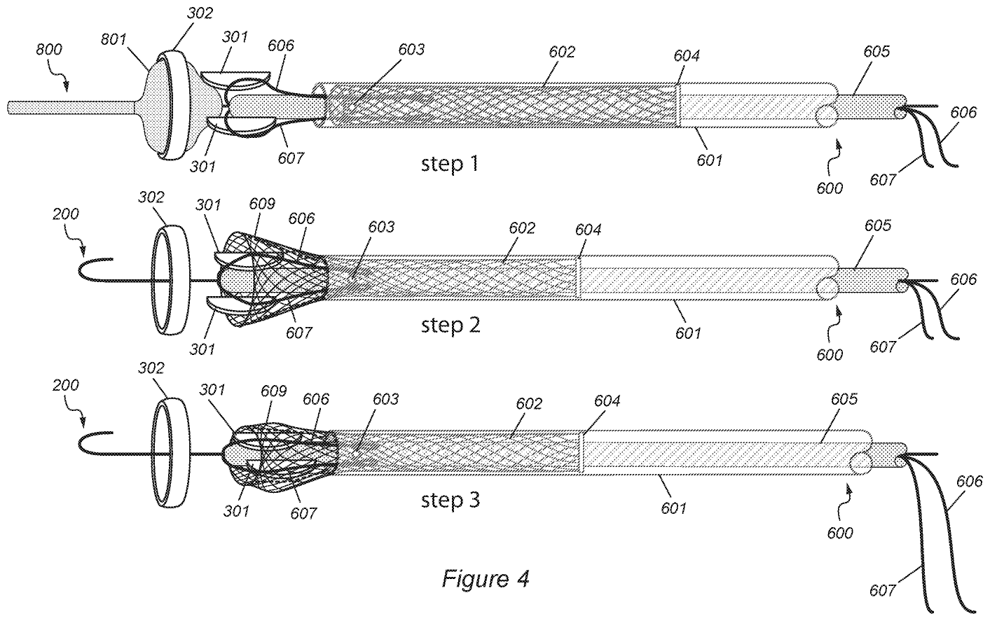

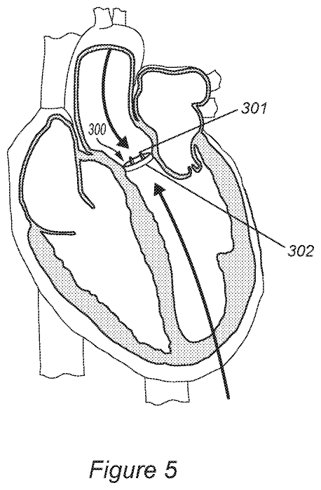

While various embodiments of the device incorporate memory mesh, in some embodiments, the device is configured such that the shape of the leaflet capturing component can be manually manipulated through the use of one or more strings or wires. Although one of skill in the art would readily appreciate that numerous configurations of wires and strings are possible, in certain embodiments, one or more wires or strings are threaded through a lumen of the catheter, extending from the trailing end (or a region near the trailing end) through the leading end (or a region near the leading end), and attaching to the leaflet capturing component at one or more locations. For example, as demonstrated in FIG. 4, a first string 606 and second string 607 can be threaded through the device 600 and attached to the mesh 602. By manipulating the length of the strings 606 and 607, the mechanical heart valve leaflets 301 can be captured. In some embodiments, the sliding end of the leaflet capturing component, or any other slidable portion of the leaflet capturing component, is engaged with one or more strings or wires, such that it can be pushed and/or pulled along the catheter body to form a basket that can capture and remove leaflets, as described herein.

In certain embodiments, the mechanism for leaflet retrieval may be activated by a knob or dial on the delivery system allowing for manipulation of the mesh network. Merely by way of non-limiting example, the knob can be situated such that turning the knob would cinch down (or narrow) the orifice of the retrieval mesh, thereby trapping the leaflets like a Chinese finger trap. In some embodiments, the knob is situated at or near the end of the device, but it could be situated in any practical position on the device. The cinching down may be accomplished by a circumferential wire or string, the pulling of which would cinch down the orifice of the retrieval mesh and capture the leaflets.

In certain embodiments, the apparatus includes one or more movable wires and/or strings attached to the outer sheath, such that the position of the outer sheath relative to the mesh network can be adjusted (i.e. so that the mesh network can be extended beyond or housed within the sheath). These features can be used in conjunction with any of the aforementioned embodiments of the device.

The strings and/or wires integrated into the inventive devices described herein can be made of any suitable material, including but in no way limited to, platinum, nitinol, nickel-chromium, and the like.

In some embodiments, the memory mesh itself is capable of providing sufficient tension (when forming a basket) to separate the mechanical heart valve leaflets from the annulus. In these embodiments, the leaflet capturing component is allowed to adopt its basket conformation while inserted between the leaflets of the mechanical heart valve by releasing the leaflet capturing component from the sheath. The basket formed by the mesh can then be used to capture and remove the leaflets, as described above, by manipulating its shape using the attached strings or wires.

### Balloon Integration

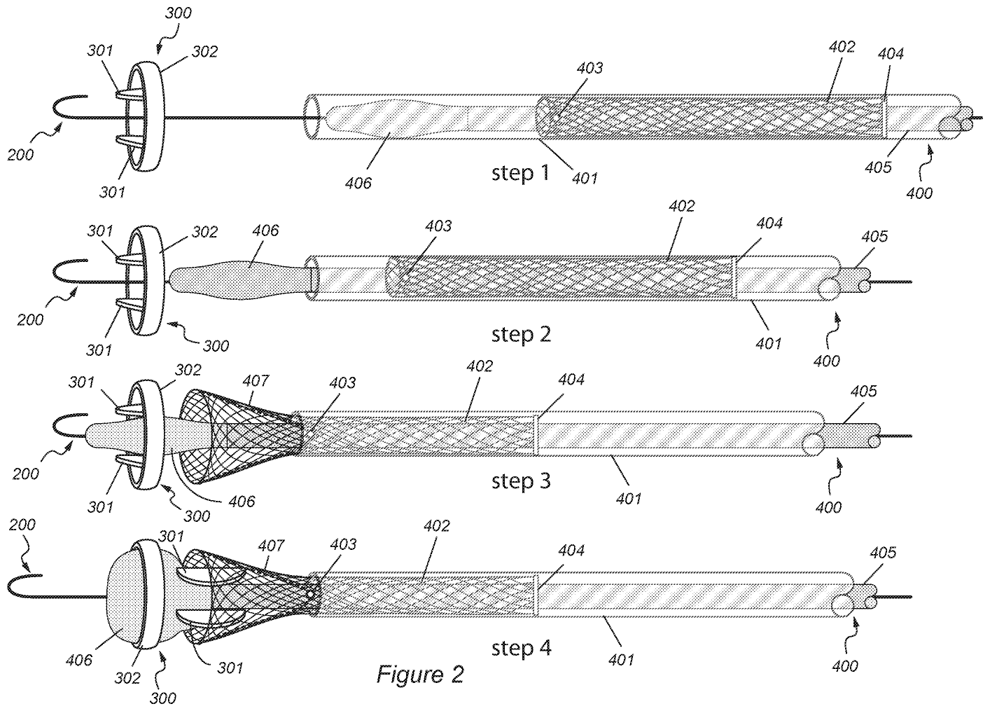

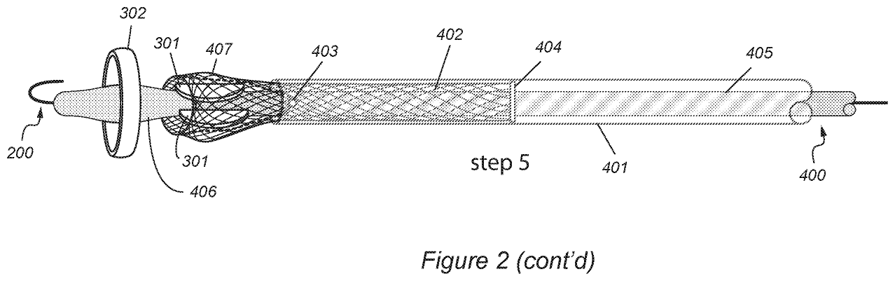

As demonstrated in FIG. 2, in certain embodiments, the device 400 can include an inflatable balloon 406 located in a fixed position on the device, such that the region of attachment of the mesh 403 always remains between the balloon 406 and the sliding end of the mesh 404 when the device is in use. As also demonstrated in FIG. 2, in some embodiments, the balloon 406 can be housed within the sheath 401, when it is not inflated. In alternative embodiments, the balloon is not configured to be housed within the sheath, yet it is still suitable for introducing into a patient, due to its dimensions and composition.

As demonstrated in FIG. 2, in some embodiments, the device includes a balloon 406 that can be inflated to a size that will apply sufficient pressure on the leaflets 301 to separate them from the annulus to which they are attached 302. As further demonstrated in FIG. 2, the balloon 406 and mesh 402 can be configured such that when the leaflets 301 are separated from the annulus 302 they are directed into the basket 407 of the device 400.

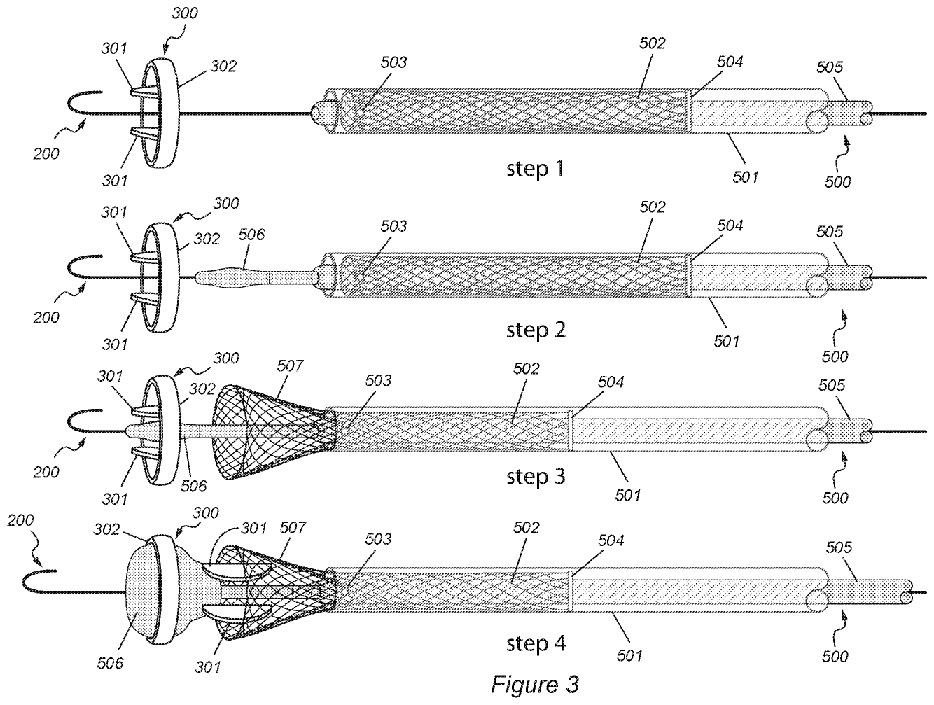

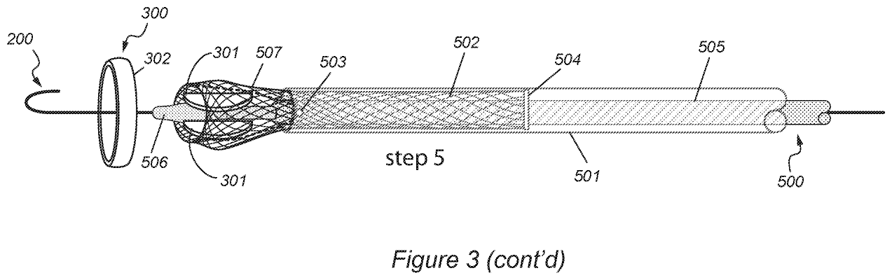

As demonstrated in FIG. 3, in certain embodiments, the device 500 includes a telescoping inner section with an integrated balloon 506. In some embodiments, the distance between the balloon 506 and the region 503 at which the mesh 502 attaches to the body of the catheter 503 can be increased or decreased as the section containing the balloon 506 is extended or retracted, respectively. In certain embodiments, the section of the device containing the balloon can be completely retracted into the catheter. In certain embodiments, the section of the device containing the balloon cannot be completely retracted into the catheter.

In certain embodiments, the devices described herein that include an integrated balloon also include one or more lumens that communicate with the balloon through one or more ports, and thereby allow for modulating the balloon's size through inflation and deflation. The devices described herein can be configured such that the balloon is inflated using any means of inflating a balloon on a catheter known in the art.

The balloons described herein can be any of a number of useful shapes. Merely by way of non-limiting examples, the balloon can be spherical, oblong, dumbbell shaped, or half dumbbell shaped. In some embodiments, the balloon is of the shape depicted in FIG. 2. One of skill in the art would readily appreciate that the balloons described herein can be made of any material suitable for their intended purpose, including, but in no way limited to, rubber, semicompliant or noncompliant plastic, and the like.

In certain embodiments of the invention in which a balloon is incorporated into the device, the balloon, lumen(s) and port(s) are configured such that the rate and extent of inflation can be regulated to allow for controlled separation of mechanical valve leaflets from a mechanical valve annulus. One of skill in the art would readily appreciate that one or more valves and/or meters could be employed in conjunction with the inventive device in order to accomplish a desired rate and/or extent of inflation and deflation of the balloon.

### Exemplary Embodiments & General Methods of Use

Turning now to more detailed descriptions of exemplary embodiments, FIG. 1 depicts a device 100 with a catheter body 105 partially housed within a mesh component 102 having an attaching end 103 and a sliding end 104. The device also includes an outer sheath 101. As shown in FIG. 1, the device 100 is configured such that the mesh 102 forms a basket 106 capable of containing mechanical heart valve leaflets 301 when the mesh 102 is advanced beyond the end of the sheath 101. The exemplary device 100 further includes a lumen for allowing it to glide along a guide wire 200 that has been inserted into a patient. The device is then positioned near the mechanical heart valve, and the mesh is advanced beyond the end of the sheath, thereby causing it to form a basket. After the mechanical heart valve leaflets have been separated from the annulus to which they were attached (by balloon catheter or otherwise), the basket is used to collect the leaflets. The basket and leaflets are then withdrawn in the direction of the trailing end of the catheter, causing the mesh to close around the leaflets in the manner of a Chinese finger trap. Finally, the device and leaflets are completely removed from the patient.

FIG. 2 depicts another embodiment of the invention 400 with a catheter body 405 and a mesh capturing component 402, which is attached near the leading end of the catheter at one end 403 and terminates in a sliding component 404 at the other end. The device 400 of FIG. 2 also includes a balloon 406 and an outer sheath 401. In practice, the device is inserted into a patient over a guide wire 200, and positioned proximate to a mechanical heart valve of interest 300, which could be in aortic, mitral, pulmonic or tricuspid position. The balloon 406 is subsequently inserted between the mechanical heart valve leaflets 301 and then inflated, thereby separating the leaflets 301 from the annulus 302 and directing them into the basket 407 of the device 400. After the leaflets 301 are deposited into the basket 407, the balloon 406 is deflated and the basket 407 is retracted in the direction of the trailing end of the catheter, thereby closing the basket 407 around the leaflets like a Chinese finger trap. The device and leaflets are then removed from the patient.

In another embodiment 500, depicted in FIG. 3, a balloon 506 is integrated on a telescoping element that can be either retracted into the body of the catheter 505 or extended therefrom. This device 500 also includes a leaflet capturing component 502 (including an attaching end 503 and a sliding end 504) as well as a sheath 501. In practice, the device 500 is inserted over a guide wire 200 into a patient. The device is positioned near the mechanical heart valve 300, and the balloon 506 is then extended until it is positioned between the mechanical heart valve leaflets 301. The basket 507 is then positioned proximal to, or in contact with, the mechanical heart valve 300 from which the mechanical heart valve leaflets 301 are to be separated. Next, the balloon 506 is inflated sufficiently to separate the leaflets 301 from the annulus 302. As shown in FIG. 3, the balloon 506 can be situated closely enough to the basket 507 that the leaflets 301 are directly deposited into the basket 507. In addition, the balloon 506 can be partially deflated and then retracted towards the body of the catheter 505, thereby nudging the leaflets into the basket 507. The basket 507 is then withdrawn toward the trailing end of the catheter, thereby closing around the leaflets 301 (as described above). Finally, the balloon 506 is deflated and the device 500 and leaflets 301 are removed from the patient.

In yet another embodiment, depicted in FIG. 4, the device 600 includes strings 606 and 607 that each begins on the trailing end of the catheter. The strings are threaded through a lumen in the body of the catheter 605 and emerge at the leading end. The strings terminate at a region of attachment to the mesh capturing component. The mesh capturing component 602 includes an attaching end 603 and a sliding end 604. This device also includes a sheath 601. In practice, the device 600 is inserted over a guide wire 200 into a patient, and positioned near the mechanical heart valve 300 of interest. A balloon catheter 800 is inserted into the patient and positioned between the mechanical heart valve leaflets 301. A basket 609 is then formed by manipulating the strings 606 and 607 of the device, such that it is positioned proximal to, or in contact with, the mechanical heart valve 300 from which the mechanical heart valve leaflets 301 are to be separated. Next, the balloon 801 is inflated sufficiently to separate the leaflets 301 from the annulus 302 and deposit them into the basket 609. The basket 609 is then retracted by manipulating the strings 606 and 607, thereby closing it around the leaflets 301 (as described above). Finally, the balloon 801 is deflated and the devices 800 and 600 and leaflets 301 are removed from the patient.

One of skill in the art would readily appreciate that, in some cases, the steps of the general methods described above can be performed in an alternative order, without negatively impacting the intended results.

### Specific Surgical Approaches

As indicated above, the devices and methods described above can be adapted for removing mechanical heart valve leaflets from various types of mechanical heart valves, situated at different anatomical locations. One of skill in the art would recognize that various directional approaches to the mechanical heart valve of interest can be used. In some embodiments, the approach is transapical. In other embodiments, the approach can be through a blood vessel (e.g. femoral artery->aorta).

In some cases in which leaflets of a mechanical aortic valve are to be removed, a device without an integrated balloon (such as the device 100 of FIG. 1) can be inserted into the heart transapically or endovascularly (i.e. via artery or vein). A balloon catheter is then used for separating the mechanical valve leaflets from the mechanical valve annulus, as depicted in FIGS. 6 and 7. One of skill in the art would readily appreciate that the two separate devices can be inserted into the patient from the same direction, or from opposing directions, from either side of any valve described herein.

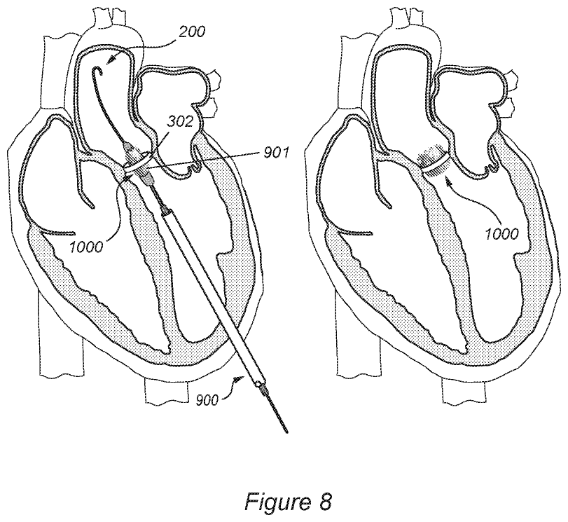

In certain cases, mechanical aortic valve leaflets can be removed according to the following procedures. First, a semi-compliant balloon is placed just proximal to the innominate artery using an endovascular approach. Next, right ventricle pacing is performed by using a standard transvenous pacemaker to stop cardiac output. Then, the semi-compliant balloon is inflated to completely occlude the distal ascending aorta, which reduces aortic regurgitation into the left ventricle. Next, the mechanical aortic valve leaflets are dislodged from the mechanical annulus, and retrieved using any of the devices and methods described herein. After the leaflets have been dislodged and removed, a new heart valve can be implanted into the remaining mechanical annulus using a standard technique (e.g. by the approach demonstrated in FIG. 8). Catheters suitable for accomplishing this aspect of the invention are well known in the art. In some embodiments, the valve replacement is accomplished by using a balloon catheter typically used for transcatheter aortic valve replacement (TAVR). One of skill in the art would readily appreciate that any balloon catheter of the appropriate dimensions and with the appropriate features, such as those referenced or described in U.S. patent application Ser. No. 13/666,700, which is incorporated herein by reference in its entirety, can be used. In some embodiments, the replacement valve is a bioprosthetic valve. Any structurally suitable bioprosthetic valve could be utilized to replace the mechanical valve in this “valve-in-valve” approach, including, but in no way limited to, the SAPIEN valve (Edwards), CoreValve (Medtronic), Portico (St Jude Medical), and the like. In preferred embodiments, the replacement valve selected doesn't require the patient to take anticoagulant medications in the long-term, and preferably at all.

In certain embodiments, a specialized balloon that functions like a valve can be used in place of the semi-compliant balloon. In these cases, the balloon functions like an intraortic balloon pump (IABP), inflating in diastole and deflating promptly for systole. Using this strategy, blood is prevented from returning to the left ventricle from the aorta, after the mechanical heart valve leaflets have been removed, and before a replacement valve is positioned. However with the “IABP”-type ascending aortic balloon, the cardiac output is maintained. Furthermore, the IABP balloon is placed in the proximal descending thoracic aorta, while this balloon is placed in the distal ascending aorta. This general strategy/method can be utilized in any procedure in which any device for capturing mechanical heart valve leaflets described herein (or any other device with a similar function) is used.

In addition to the aforementioned devices and methods, the present invention also teaches a kit directed to transcatheter removal of mechanical heart valve leaflets. The kit is an assemblage of one or more materials and components, including at least one of the devices described herein.

The exact nature of the components configured in the inventive kit depends on its intended purpose. In some embodiments, the kit is configured for dislodging and subsequently removing mechanical heart valve leaflets from a patient, and therefore it includes one or more of the devices described above and suited for that purpose. In some embodiments, the kit further includes a device that can be used to implant a bioprosthetic valve within a mechanical heart valve annulus, as described above. In some embodiments, the replacement valve, which may include any replacement valve described herein (or the like), is included in the kit. In some embodiments, the kit includes a guide wire of a size appropriate to interact with the device with which it is intended to be used.

In one embodiment, the kit is configured particularly for the purpose of treating mammalian subjects. In another embodiment, the kit is configured for the purpose of treating human subjects. In another embodiment, the kit is configured for treating adolescent, child, or infant human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as removing mechanical heart valve leaflets, dislodging mechanical heart valve leaflets from a mechanical heart valve annulus, implanting a bioprosthetic valve, or combinations thereof. Optionally, the kit also contains other useful components, such as materials used for surgical preparation and catheter placement.

The materials or components assembled in the kit are typically contained in suitable packaging material(s). As employed herein, the phrase “packaging material” refers to one or more physical structures used to house the contents of the kit, which can include one or more of the devices described herein, depending upon the particular desired application. The packaging material is constructed by well-known methods, preferably to provide a sterile, contaminant-free environment. As used herein, the term “package” can refer to plastic, paper, foil, and the like, or similar materials capable of holding the individual kit components. The packaging material generally has an external label which indicates the contents and/or purpose of the kit and/or its components.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### EXAMPLES

### Example 1

### Surgical Procedure

After induction of anesthesia, and sterile preparation, percutaneous femoral access with a 12 F sheath is performed. The subject is heparinized with an activated clotting time (ACT) over 250 sec. The wire is advanced under fluoroscopic guidance into the ascending aorta. An intra-aortic ballon pump (IABP)-type balloon is advanced and positioned in the distal ascending aorta (proximal to the orifice of the innominate artery). Subsequently a right ventricular pacing lead is placed through the right internal jugular vein into the right ventricle, and tested for appropriate sensing and pacing.

Concomitantly an incision is performed on the 5^{th} intercostal space in the mid-clavicular line, cutting the intercostal muscles, and entering the left pleural cavity. Next, the pericardium over the left ventricular apex is opened, and four pledgeted sutures are placed at the site of entry. Next an 18 gauge needle and 0.035 wire is inserted and advanced through the mechanical valve. The needle is withdrawn and the inventive sheath (101 on FIG. 1) is inserted in the left ventricular apex.

Next, the rapid ventricular pacing is initiated, thereby reducing cardiac output. Then the IABP-type balloon is inflated in the ascending aorta, and the dumbbell-shaped balloon (406 on FIG. 1) is inserted into the mechanical valve with the leaflet-capturing mesh (407 on FIG. 1) abutting the most proximal side of the valve. Subsequently, the balloon in inflated, thereby dislodging the leaflets that are captured by the leaflet-capturing mesh device. At this time, the balloon and the leaflets are removed from the subject, leaving the sheath (401 on FIG. 1) in place. The prepared transcatheter aortic valve device using a bioprosthesis is then inserted into transapical sheath and deployed within the mechanical valve annulus (302 on FIG. 1). At this time, the ventricular pacing is stopped, the IABP-type balloon is deflated and withdrawn, and normal ventricular ejection through the newly implanted bioprosthesis is initiated. At this time, echocardiographic function of the valve is assessed, and aortic angiogram in the ascending aorta is performed through a pigtail inserted through the femoral access. Hemodynamics of the newly placed aortic valve are assessed, along with need for re-balloon on the newly placed bioprosthesis. At this time, the transapical and transfemoral sheath and wire are removed. Transapical access is repaired by ligating the pledgeted sutures, closing the incision in three layers, and leaving a drain in the left chest cavity. The transfemoral access is repaired percutanously using an existing suture-mediated closure device, such as proglide (Abbott company), and heparin is reversed using protamine.

Although the use of the IABP-type balloon pump is described in the context of a specific type of leaflet retrieval device, the IABP-type balloon pump could also be used in a like manner with any of a number of leaflet retrieval devices with a similar effect.

### Example II

### Alternative Route

After induction of anesthesia, and sterile preparation, a right ventricular pacing lead is placed through right internal jugular vein into the right ventricle, and tested for appropriate sensing and pacing.

Concomitantly an incision is performed on the 2nd right intercostal parasternal space, cutting the intercostal muscles, and entering the right pleural cavity. Next, the pericardium over the ascending aorta is opened, and four pledgeted sutures are placed on the aorta at the site of entry. Concomitantly, percutaneous femoral access with a 12 F sheath is performed. The subject is heparinized with an activated clotting time (ACT) over 250 sec. The wire is advanced under fluoroscopic guidance into the ascending aorta. The IABP-type balloon is advanced and parked in the transverse aorta (distal to the entry point of the inventive device). Next an 18 gauge needle and 0.035 wire is inserted from right thoracotomy incision and advanced through the mechanical valve. The needle is withdrawn and the inventive sheath (401 on FIG. 2) is inserted in the ascending aorta aiming at the valve.

Next, the rapid ventricular pacing is initiated, thereby reducing cardiac output. Then the IABP-type balloon is inflated in the transverse aorta, and the dumbbell-shaped balloon (406 on FIG. 2) is inserted into the mechanical valve with the leaflet-capturing mesh (407 on FIG. 2) abutting the most distal side of the valve. Subsequently, the balloon in inflated, thereby dislodging the leaflets in the direction so they are captured by the leaflet-capturing mesh device. At this time, the balloon and the leaflets are removed from the subject, leaving the sheath (401 on FIG. 2) in place. The prepared transcatheter aortic valve device using a bioprosthesis is now inserted into the transapical sheath and deployed within the mechanical valve annulus (302 on FIG. 2). At this time, the ventricular pacing is stopped, the IABP-type balloon is deflated and withdrawn, and normal ventricular ejection through the newly implanted bioprosthesis is initiated. At this time, echocardiographic function of the valve is assessed, and aortic angiogram in the ascending aorta is performed through a pigtail inserted through the right thoracotomy access. Hemodynamics of the newly placed aortic valve are assessed, along with need for re-balloon on the newly placed bioprosthesis. At this time, the trans-arotic and transfemoral sheath and wire are removed. Trans-aortic access is repaired by ligating the pledgeted sutures, closing the incision in three layers, and leaving a drain in the right chest cavity. The transfemoral access is repaired percutanously using existing suture-mediated closure devices such as proglide (Abbott company), and heparin is reversed using protamine.

What is claimed is:

1. An apparatus for transcatheter removal of mechanical heart valve leaflets from a subject, comprising:
a catheter, comprising:
a first catheter end;
a second catheter end; and
an elongated catheter body;
a sheath, comprising:
a first sheath end;
a second sheath end; and
an elongated sheath body;
a leaflet capturing component, comprising:
an attaching end for attaching to a region near the first catheter end;
an elongated leaflet capturing body; and
a sliding end; wherein (1) the catheter, sheath and leaflet capturing component are situated concentrically, such that the sheath is the outermost component and the catheter is the innermost component; (2) the sliding end is configured to traverse the elongated catheter body; and (3) when the sliding end is advanced in the direction of the attaching end, a section of the leaflet capturing component is extended beyond the first sheath end, thereby forming a basket.

2. The apparatus of claim 1, wherein the leaflet capturing component comprises mesh.

3. The apparatus of claim 2, wherein the mesh is made of a material selected from the group consisting of nitinol, semicompliant platinum, nickel-chromium, and combinations thereof.

4. The apparatus of claim 1, further comprising an inflatable balloon.

5. The apparatus of claim 4, wherein the inflatable balloon is located between the attaching end of the leaflet capturing component and the first catheter end.

6. The apparatus of claim 5, wherein the inflatable balloon is asymmetrical when inflated.

7. The apparatus of claim 6, wherein the inflatable balloon has an asymmetrical half dumbbell shape when inflated

8. The apparatus of claim 5, further comprising a means for inflating the balloon.

9. The apparatus of claim 5, further comprising a means for causing the leaflet capturing component to form a basket.

10. The apparatus of claim 5, wherein the catheter is telescoping, such that its overall length can be adjusted.

11. A system, comprising:
(a) an apparatus for transcatheter removal of mechanical heart valve leaflets from a subject, comprising:
a catheter, comprising:
a first catheter end;
a second catheter end; and
an elongated catheter body with an elongated lumen traversing the long axis of said elongated catheter body;
a sheath, comprising
a first sheath end;
a second sheath end; and
an elongated sheath body;
a leaflet capturing component, comprising:
an attaching end for attaching to a region near the first catheter end;
an elongated leaflet capturing body; and a sliding end; wherein (1) the catheter, sheath and leaflet capturing component are situated concentrically, such that the sheath is the outermost component and the catheter is the innermost component; (2) the sliding end is configured to traverse the elongated catheter body; and (3) when the sliding end is advanced in the direction of the attaching end, a section of the leaflet capturing component is extended beyond the first sheath end, thereby forming a basket; and
(b) a guidewire configured to traverse the elongated lumen of the catheter body.

12. A method, comprising:
introducing the apparatus of claim 1 into the heart of a subject having a mechanical heart valve, wherein the mechanical heart valve comprises mechanical heart valve leaflets attached to a mechanical heart valve annulus;
introducing a balloon of a balloon catheter between the mechanical heart valve leaflets of the mechanical heart valve;
inflating the balloon until the mechanical heart valve leaflets are separated from the annulus of the mechanical heart valve;
forming a basket with the leaflet capturing component of the apparatus of claim 1 by advancing the sliding end of the leaflet capturing component in the direction of the attaching end of the leaflet capturing component;
enveloping the separated mechanical heart valve leaflets with the basket;
withdrawing the basket in the direction of the second end of the catheter, thereby substantially containing the mechanical heart valve leaflets within the basket; and
removing the apparatus of claim 1 and the mechanical heart valve leaflets from the subject.

13. The method of claim 12, wherein the apparatus of claim 1 is introduced into the subject's heart through a blood vessel.

14. The method of claim 12, wherein the apparatus of claim 1 is introduced into the subject's heart transapically.

15. The method of claim 12, further comprising placing a replacement heart valve in the mechanical heart valve annulus by using a balloon catheter, after the mechanical heart valve leaflets have been removed.

16. The method of claim 15, wherein the replacement heart valve is a bioprosthetic valve.

17. The method of claim 12, wherein the apparatus of claim 1 and the balloon catheter are inserted into the subject's body such that they approach the mechanical heart valve from opposite directions.

18. The method of claim 12, wherein the apparatus of claim 1 and the balloon catheter are inserted into the subject's body such that they each approach the mechanical heart valve from the same direction.

19. The method of claim 12, wherein the mechanical heart valve is located in the position of a heart valve selected from the group consisting of an aortic valve, a mitral valve, a pulmonic valve and a tricuspid valve.

20. A method, comprising:
introducing the apparatus of claim 4 into the heart of a subject having a mechanical heart valve comprising mechanical heart valve leaflets attached to a mechanical heart valve annulus;
introducing the balloon of the apparatus of claim 4 between the mechanical heart valve leaflets of the mechanical heart valve;
inflating the balloon of the apparatus of claim 4 until the mechanical heart valve leaflets are separated from the annulus of the mechanical heart valve;
forming a basket with the leaflet capturing component of the apparatus of claim 4 by advancing the sliding end of the leaflet capturing component in the direction of the attaching end of the leaflet capturing component;
enveloping the mechanical heart valve leaflets with the basket;
withdrawing the basket in the direction of the second end of the catheter, thereby substantially containing the mechanical heart valve leaflets within the basket;
deflating the balloon; and
removing the apparatus of claim 4 and the mechanical heart valve leaflets from the subject.

21. The method of claim 20, wherein the mechanical heart valve is located in the position of a heart valve selected from the group consisting of an aortic valve, a mitral valve, a pulmonic valve and a tricuspid valve.

22. The method of claim 20, further comprising placing a replacement heart valve in the mechanical heart valve annulus by utilizing a balloon catheter, after the mechanical heart valve leaflets have been removed.

23. The method of claim 22, wherein the replacement heart valve is a bioprosthetic valve.

24. A kit, comprising:
(a) an apparatus for transcatheter removal of mechanical heart valve leaflets from a subject, comprising:
a catheter, comprising:
a first catheter end;
a second catheter end; and
an elongated catheter body with an elongated lumen traversing the long axis of said elongated catheter body;
a sheath, comprising
a first sheath end;
a second sheath end; and
an elongated sheath body;
a leaflet capturing component, comprising:
an attaching end for attaching to a region near the first catheter end;
an elongated leaflet capturing body; and a sliding end; wherein (1) the catheter, sheath and leaflet capturing component are situated concentrically, such that the sheath is the outermost component and the catheter is the innermost component; (2) the sliding end is configured to traverse the elongated catheter body; and (3) when the sliding end is advanced in the direction of the attaching end, a section of the leaflet capturing component is extended beyond the first sheath end, thereby forming a basket;
(b) a guidewire configured to traverse the elongated lumen of the catheter body; and optionally
(c) instructions for the use of the apparatus for removing mechanical heart valve leaflets from a subject.
